(19) United States
(12) Patent Application Publication
(10) Pub. No.: US 2014/0088607 A1
(43) Pub. Date: Mar. 27, 2014

(54) MOBILE KYPHOSIS ANGLE MEASUREMENT

(71) Applicant: Chris P. Recknor, Gainesville, GA (US)

(72) Inventor: Chris P. Recknor, Gainesville, GA (US)

(21) Appl. No.: 13/629,586

(22) Filed: Sep. 27, 2012

(51) Int. Cl. A61B 5/103

(52) U.S. Cl. 606/102

(57) ABSTRACT

A hand-held mobile device configured to measure spinal curves for detecting and analyzing kyphosis. The measuring device is equipped with a detector that can sense the angle of at certain points of the spine by holding an edge of the device against the spine and detecting the angle of inclination of the device. The device can interface with a server for collecting, storing and analyzing several data points collected over time, and providing diagnoses and remedial measures.

### CROSS-REFERENCE TO RELATED APPLICATIONS

This is a utility patent application being filed in the United States as a non-provisional application for patent under Title 35 U.S.C. §100 et seq. and 37 C.F.R. §1.53(b). This application incorporates U.S. Pat. No. 7,556,045 by reference in its entirety.

### BACKGROUND

1. Field of Invention

This disclosure relates generally to the field of medical evaluation tools and, more particularly, to a system, method and device for measuring and evaluating kyphosis in a patient.

2. Description of Related Art

In today's high paced, high-tech world in which most people wear multiple hats and keep a few balls in the air during the juggling act that they call life, it is quite common that many important things fall by the wayside as people are dragged around all day having to deal with the urgent issues. One of the most important things that often times falls victim to neglect, crowded out by the “urgent” things in life, is a person's health. In the course of a busy day, week, month, quarter, etc., carving out a chunk of time to visit the doctor's office for a much needed examination, check-up or adjustment may simply not be “convenient”. Even though the wisdom of the old adage “an ounce of prevention is worth a pound of cure” is undeniable, many people find themselves repeatedly neglecting their self maintenance and health checks, oftentimes until it is too late to resolve the issue.

According to the National Institute of Health, physical functional decline is often the determining factor that leads to loss of independence in older persons. Identifying risk factors for physical disability may lead to interventions that may prevent or delay the onset of functional decline. In a paper published by the NIH in May of 2005 entitled Hyperkyphotic Posture and Poor Physical Functional Ability in Older Community-Dwelling Men and Women: The Rancho Bernardo Study, the authors conducted a study to determine the association between hyperkyphotic posture and physical functional limitations. In general, a physically active lifestyle should be promoted to maintain good health; however, there may be certain populations that are at particular risk for poor physical function and, that could potentially benefit from early intervention. If risk factors for poor physical function can be identified before the onset of disability, targeted interventions for those at risk may potentially prevent or delay the onset of dependence.

Kyphosis is, by definition, both an anatomical description and a condition. As a feature of the spine, kyphosis is the angle of the thoracic curve. When the angle of the thoracic curve exceeds the normal upper limit of 40-42°, this condition is called hyperkyphosis. Hyperkyphosis, or an increased thoracic curvature, is commonly observed in older persons and may be an important determinant of poor physical function. Of the studies that have investigated the association between hyperkyphosis and physical function, many have found a strong association between having excess kyphosis and poor physical functioning, by self-reported and objective measures.

There are several causes for hyperkyphosis. Some of these causes include osteoporosis leading to vertebral compression fracture, habitual poor posture, degenerative diseases of the intervertebral discs, sarcopenia, intervertebral ligament contraction and genetics.

In younger populations, normal kyphosis angles range between 20-40 degrees. However, in older adults, the mean kyphosis angle is about 48-50 degrees in women and about 44 degrees in men. Cross-sectional studies have found that the oldest age groups have the most pronounced increases in kyphosis. The following table provides reported mean thoracic kyphosis angles based on age groups:

TABLE 1

| Mean thoracic kyphosis angles | Age Group |
|---|---|
| 26 degrees | 20s |
| 53 degrees | 60-74 years of age |
| 66 degrees | older than 75 years of age |

Some studies have shown that the mean thoracic angle increased approximately 3 degrees per decade. Despite the vast amounts of information and research accenting the link between increased kyperkyphosis and declining physical capabilities, there has not been a uniformly accepted threshold for defining either hyperkyphosis or normal thoracic spine changes associated with aging. Further, the multiple methods that have been promulgated for measuring kyphosis tend to produce different results.

A common technique used to measure kyphosis is with radiographic images, or X-rays taken of the patient's spine from a side view. From the X-ray image, a skilled artisan can simply observe the curvature of the spine and using various tools, identify the angles and arc radius of the curves. Although the use of radiographic imaging is certainly an accurate technique to measure kyphosis, it can be rather expensive, time consuming and require radiating the patient. Thus, other techniques have been developed to measure the kyphosis of a patient.

Some of the techniques that are commonly used include visual inspections and measuring/tracking height loss. However, visual inspections are exceedingly prone to inaccuracies and, while height loss, such as 2 centimeters or more, is correlated with increased incidence of vertebral compression fractures (VCF), there are known inconsistencies with this measurement.

Another technique to measure kyphosis is the block method. In the block method, the patient lays flat on an examination table with his or her neck in a neutral position. The kyphosis is measured by counting the number of 1.7 cm blocks that can be inserted between the patient's head and the examination table while lying in this position.

The Cobb angle, which is named after the American orthopedic surgeon John Robert Cobb, was originally used to measure coronal plane deformity on antero-posterior plain radiographs in the classification of scoliosis. It has subsequently been adapted to classify sagital plane deformity, especially in the setting of traumatic thoracolumbar spine fractures. In such use, the Cobb angle is defined as the angle formed between a line drawn parallel to the superior endplate of one vertebra above the fracture and a line drawn parallel to the inferior endplate of the vertebra one level below the fracture. FIG. 1 is a diagram illustrating the measurement of the Cobb angle in an exemplary spine. The Cobb angle between to vertebra along a spine is determined by projecting a line perpendicular to the upper endplate of an uppermost vertebra V2 and the lower endplate of the lowest vertebra V3. The angle that these two lines form with a vertical line, angle A and angle B, combine to determine the Cobb angle which is equal to A+B.

More recently, the use of inclinometers have been used to make these measurements rather than taking the measurements from and X-ray. FIG. 2 is diagram illustrating the use of an inclinometer in measuring kyphosis angles, or Cobb angles in the sagital plane. This figure has been adapted from the article entitled Clinical measurement of the thoracic kyphosis. A study of the intra-rater reliability in subjects with and without shoulder pain, written by Jeremy S. Lewis and Rachel E. Valentine and published on the biomedcentral website under BMC Musculoskeletal Disorders. An inclinometer 202 is used to measure the angle α at the juncture of vertebra T1 and T2, and the angle β at the juncture of vertebra T12 and L1. The sum of these two measures is the kyphosis angle.

FIG. 3 is a diagram illustrating an exemplary technique for performing a complete kyphosis measurement for a patient. The illustrated technique involves taking angle measurements at the Thoracic Curve 302 between vertebrae C7 and T1, the Lordotic Curve 304 between vertebrae T12 and L1 and at the midpoint between the lumbar curve and the sacral curve or the sacral midpoint 306 between vertebrae L5 and S1. From these measurements, the curve of the kyphosis angles are measured and the curvature of the spine can be projected.

### BRIEF SUMMARY

The present disclosure presents embodiments of a kyphosis measuring system and apparatus the enables measurements of the kyphosis angles of a patient's spine, records the measurements, provides analysis and diagnoses based on the measurements, and can provide remedial measures to retard the progression of the kyphosis.

One embodiment of the kyphosis measuring system includes an apparatus for measuring and analyzing spinal angles. The apparatus includes a detector that identifies the position of the apparatus; a calibrator that when actuated, calibrates the position of the apparatus to a known orientation; and a user interface executed by a processor in the apparatus that is configured guide a user in performing angular measurements of a patient's spine.

The detector may include a gyroscope, one or more accelerometers, GPS technology and or a combination of two or more of these technologies coupled with other technologies and software algorithms to assist in mathematically determining or calculating angular measurements of a spine.

The user interface includes software, running on a processor in the measuring device, that operates to perform various functions. One such function is to calibrate the apparatus. In taking measurements of a patient's spine, the device must be able to provide angular measurements based on a known orientation of the device. In an exemplary embodiment, the calibration is performed by aligning an edge of the apparatus with a surface that has a known angle or orientation, such as a vertical surface (i.e., a wall) or a horizontal surface (i.e., a table), however, any surface at a known angle could be used in the calibration process.

The apparatus can then be used to take one or more measurements at a location along the spine. For instance, measurements may be taken at particular locations along the spine and the angle of measurement can be used to determine the curvature of the spine. In some embodiments, the apparatus may then display a bone safety evaluation showing an analysis and diagnosis of the spine based on the measurements. The bone safety report may include a graphical view showing the curvature of the spine, areas of concern, areas that fall outside of normal parameters, etc.

The apparatus, in some embodiments is self contained but in other embodiments, the apparatus may interface to a server and transmits the spinal angular measurements to the server. In such embodiments, the server may conduct an analysis of the measurements and transmit a bone safety evaluation or other report back to the apparatus.

In some embodiments, the apparatus and/or the server can analyze the spinal angular measurements and generate remedial actions based at least in part on the spinal angular measurements. The remedial actions can be used to automatically adjust therapeutic equipment or appliances that the patient may be using to control or retard the progression of kyphosis.

Advantageously, embodiments of the kyphosis measuring apparatus and system can help to more quickly and efficiently monitor and track degeneration in patients. Some embodiments may even allow a patient to take measurements and have the measurements automatically transmitted to a physician without the patient having to take time to travel to the physician's office. Such capabilities help to alleviate various needs in the art and allow for the identification of problems and the implementation of preventative measures with minimal inconvenience to the busy person that simply does not have time to visit the physician or, for elderly people that have difficulty in arranging and travelling to the physician's office.

### BRIEF DESCRIPTION OF THE SEVERAL VIEWS OF THE DRAWING

FIG. 1 is a diagram illustrating the measurement of the Cobb angle in an exemplary spine.

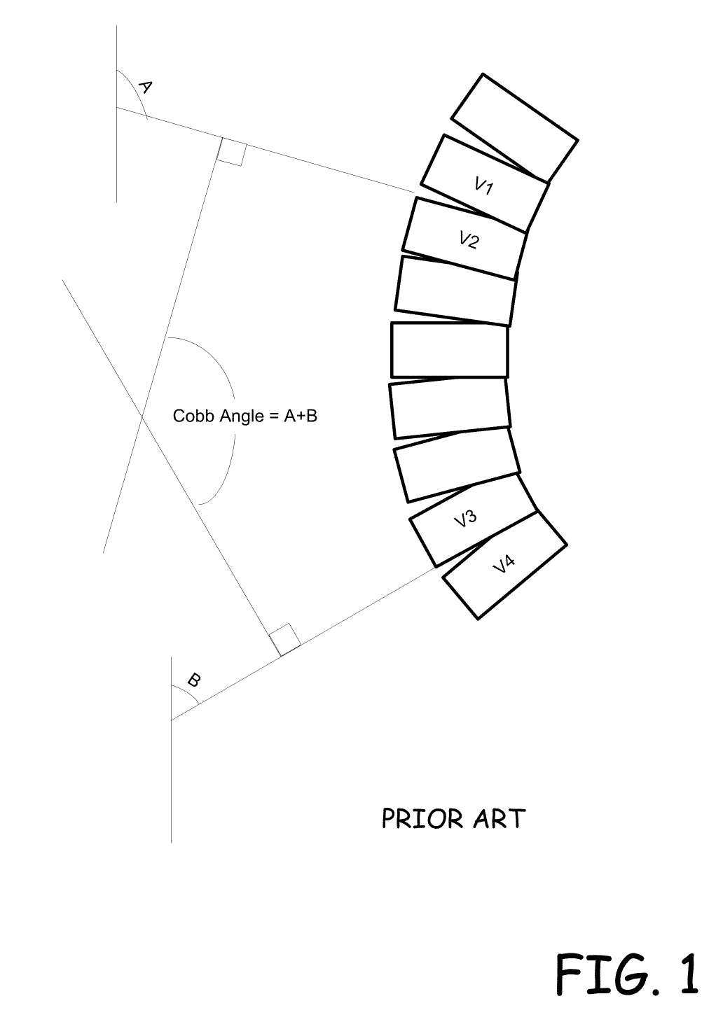

FIG. 2 is diagram illustrating the use of an inclinometer in measuring kyphosis angles, or Cobb angles in the sagital plane.

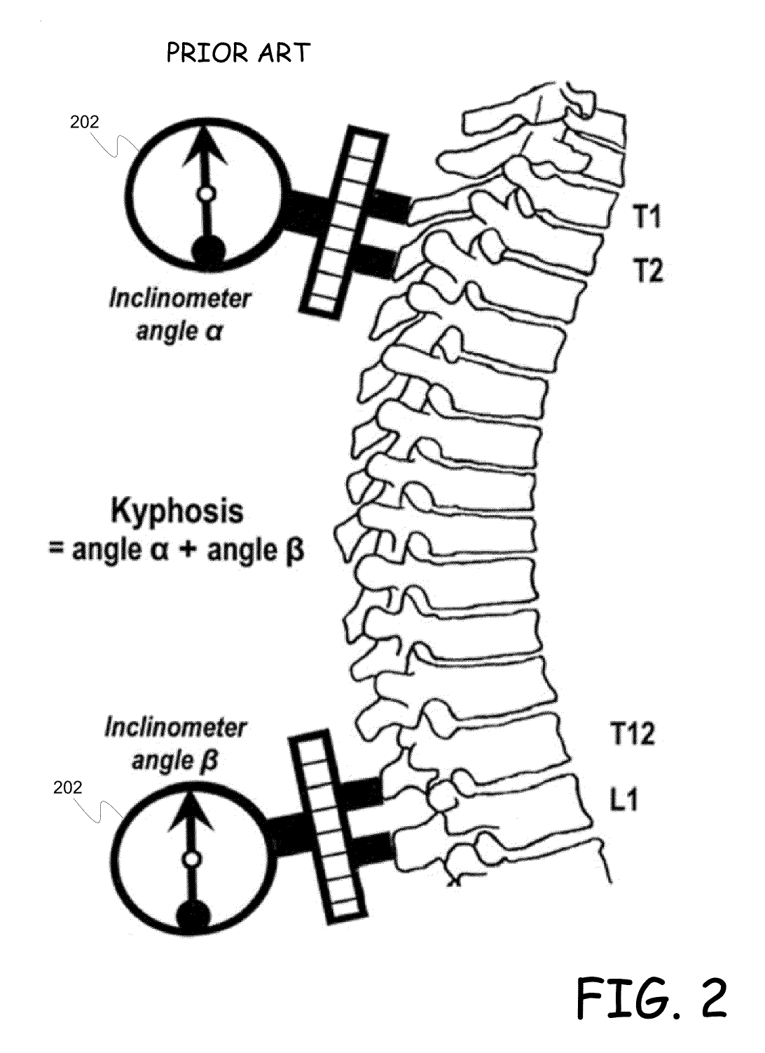

FIG. 3 is a diagram illustrating an exemplary technique for performing a complete kyphosis measurement for a patient.

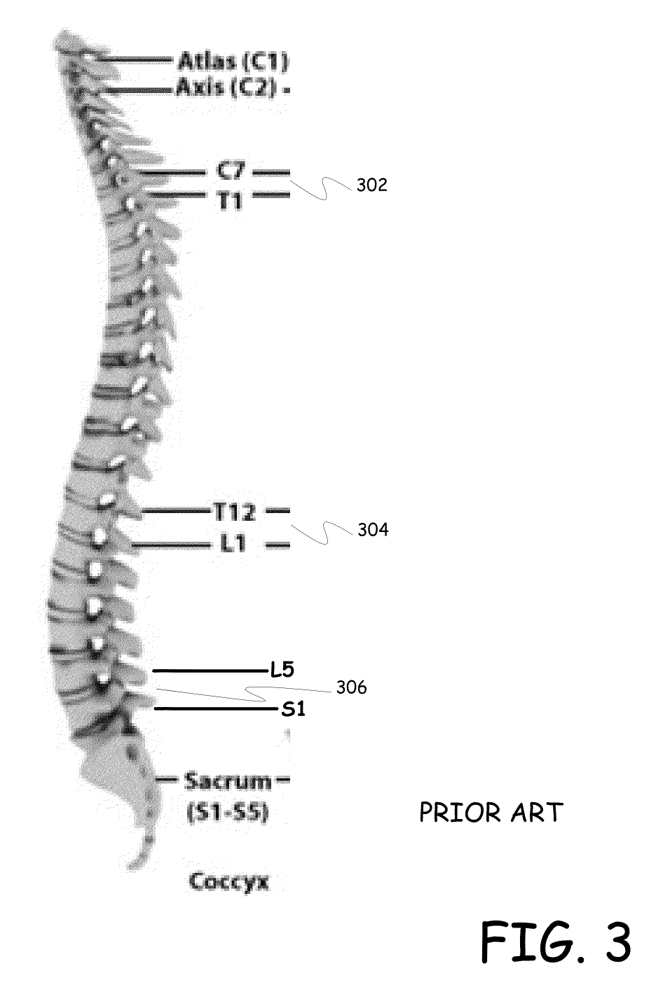

FIG. 4 is a general and functional block diagram of the components of an exemplary system or sub-system that can operate as a controller or processor that could be used as the platform of various embodiments of the KMS, KAMD or components of either for implementing the various embodiments.

FIG. 5 is a block diagram illustrating exemplary components of the KMS and communication paths associated with the illustrated components.

FIG. 6 is a flow diagram illustrating the exemplary flow of operation for an embodiment of the KMS.

FIG. 7 is a flow diagram illustrating the operation of an exemplary embodiment of a KAMD.

FIG. 8A is a screen shot of an exemplary KAMD in operation.

FIG. 8B is a screen shot of an exemplary KAMD in operation.

FIG. 8C is a screen shot of an exemplary KAMD in operation.

FIG. 8D is a screen shot of an exemplary KAMD in operation.

FIG. 8E is a screen shot of an exemplary KAMD in operation.

FIG. 8F is a screen shot of an exemplary KAMD in operation.

FIG. 8G is a screen shot of an exemplary KAMD in operation.

FIG. 8H is a screen shot of an exemplary KAMD in operation.

### DETAILED DESCRIPTION OF EMBODIMENTS

The present disclosure is directed towards a system and method for measuring spine curvature for patients, recording and analyzing the measurements, tracking changes in the curvature of the patient's spine over time, and providing preventative actions to be taken to prevent further progression or to retard progression of kyphosis. Various embodiments of a kyphosis measuring system, as well as features, aspects and elements of various embodiments are presented herein and, although some of the features, aspects and elements may be absent in some embodiments, it should be appreciated that any of the embodiments, as well as variations thereof, may include some or all of the features, aspects and elements presented in connection with other embodiments, as well as other non-described features, aspects and elements. Further, methods for utilizing the kyphosis measuring system are also presented.

Throughout this description, the following acronyms will be utilized: KMS refers to a kyphosis measuring system; KAMD refers to a kyphosis angle measuring device; and KAMA refers to kyphosis angle measuring application that can be executed by a KAMD.

The present description presents a general environment in which the KMS and KAMD or components thereof can be implemented. It should be appreciated that the KMS and KAMD, as well as components thereof can be implemented in any combination of hardware and/or software components. Next, configurations of the KMS are presented followed by further details of the components of the KMS, including the KAMD and an exemplary KAMA. Finally, uses of the KMS and the KAMD in the measurements, analysis and treatment of kyphosis are presented.

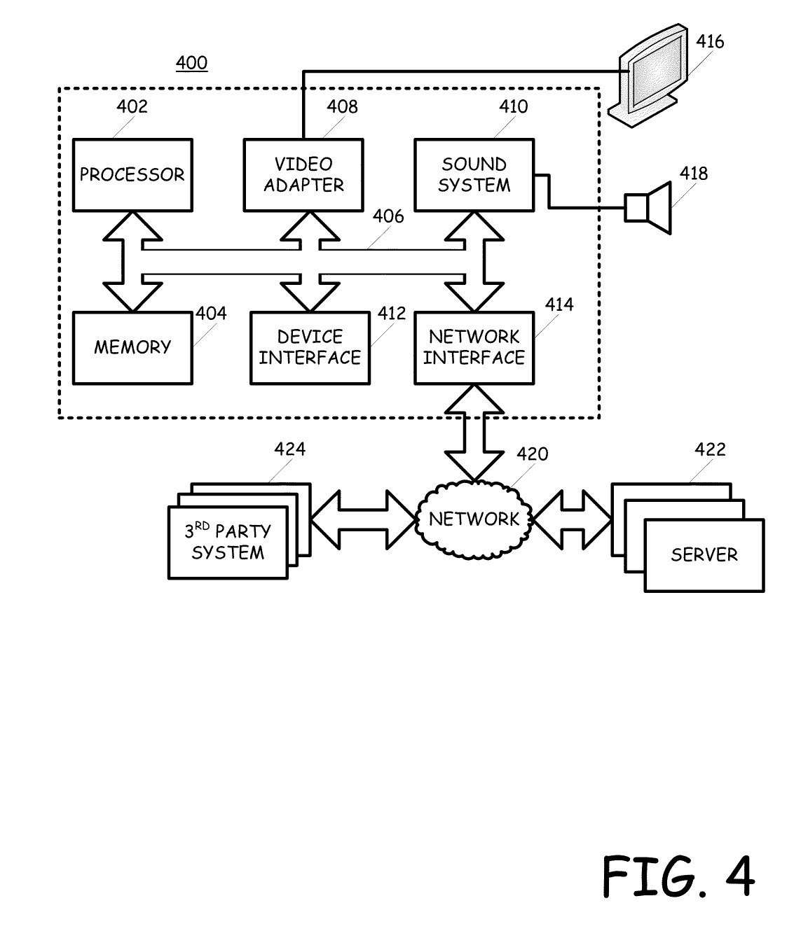

FIG. 4 is a general and functional block diagram of the components of an exemplary system or sub-system that can operate as a controller or processor that could be used as the platform of various embodiments of the KMS, KAMD or components of either for implementing the various embodiments. It will be appreciated that not all of the components illustrated in FIG. 4 are required in all embodiments of the KMS or KAMD or components thereof but, each of the components are presented and described in conjunction with FIG. 4 to provide a complete and overall understanding of the components. The controller can include a general computing platform 400 illustrated as including a processor/memory device 402/404 that may be integrated with each other or, communicatively connected over a bus or other wired or wireless interface 406. The processor 402 can be a variety of processor types including microprocessors, micro-controllers, programmable arrays, custom IC's etc. and may also include single or multiple processors with or without accelerators or the like. The memory element of 404 may include a variety of structures, including but not limited to RAM, ROM, magnetic media, optical media, bubble memory, FLASH memory, EPROM, EEPROM, etc. The processor 402, or other components in the controller may also provide components such as a real-time clock, analog to digital convertors, digital to analog convertors, etc. The processor 402 also interfaces to a variety of elements including a control interface 412, a display adapter 408, an audio adapter 410, and network/device interface 414. The control interface 412 provides an interface to external controls, such as sensors, actuators, a keyboard, a mouse, a pin pad, an audio activated device, as well as a variety of the many other available input and output devices or, another computer or processing device or the like. The display adapter 408 can be used to drive a variety of alert elements 116, such as display devices including an LED display, LCD display, one or more LEDs or other display devices. The audio adapter 410 interfaces to and drives another alert element 418, such as a speaker or speaker system, buzzer, bell, etc. The network/interface 414 may interface to a network 420 which may be any type of network including, but not limited to the Internet, a global network, a wide area network, a local area network, a wired network, a wireless network or any other network type including hybrids. Through the network 420, or even directly, the controller 400 can interface to other devices or computing platforms such as one or more servers 422 and/or third party systems 424 and/or web applications or cloud applications or services. A battery or power source provides power for the controller 400.

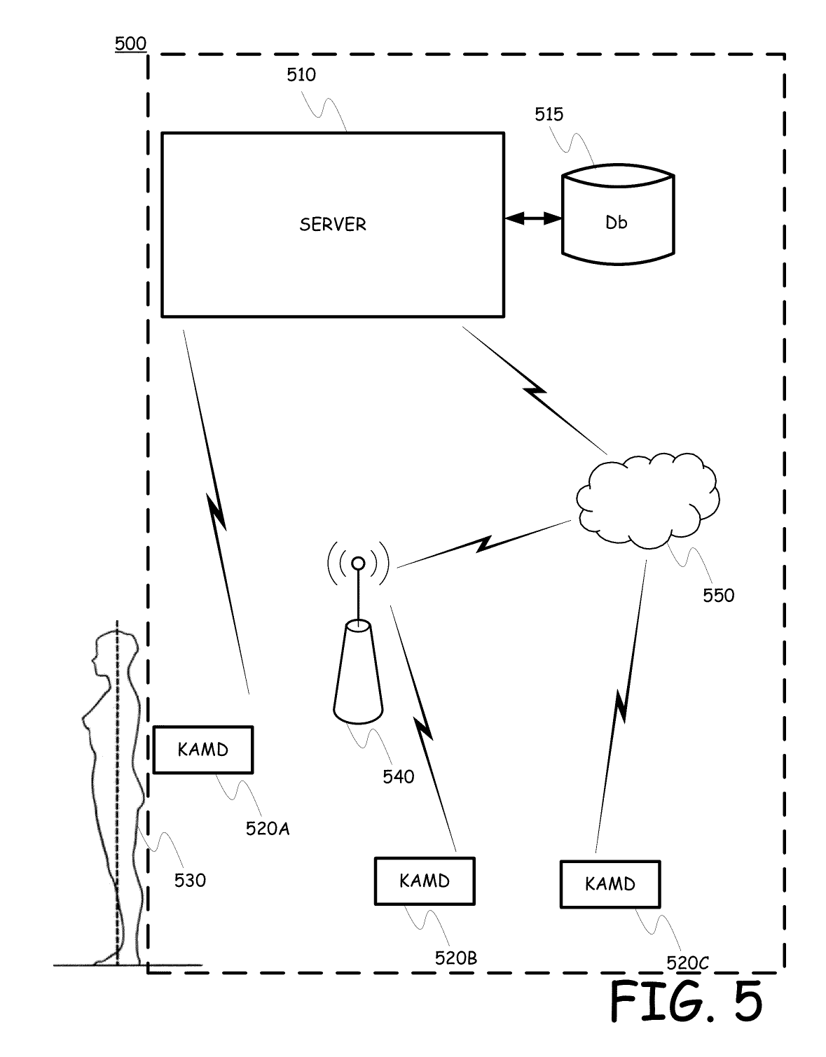

FIG. 5 is a block diagram illustrating exemplary components of the KMS and communication paths associated with the illustrated components. The KMS 500 includes a server 510, which may be a single computer or server, or multiple computers or servers, is illustrated as interfacing to a data or memory element such as database 515. Three KAMD devices 520A, 520B and 520C are illustrated in the KMS 500, however, it will be appreciated that one or more KAMDs can be utilized in the various configurations of the KMS and three KAMDs are presented only for the purpose of illustration. One KAMD 520A is shown in operation for taking angle measurements of patient 530. The KAMD 520A is illustrated as being in direct communication with the server 510. The communication between the KAMDs 520A-C and the server 510 can be accomplished using any of a variety of techniques including wireless, wired, cellular, wifi, Bluetooth, infrared, RF, optical, audio, etc. In the illustrated configuration, the KAMD 520A is directly communicatively coupled to the server 510 through a wired or wireless channel. The KAMD 520B is illustrated as communicatively coupled to the server 510 through a wireless, or at least partially wireless network which includes a wireless transceiver 540 that interfaces to the server 510 through a network 550. The KAMD 520C is illustrated as being communicatively coupled to the server 510 through a network 550.

Thus, in one embodiment, the KMS 500 may include a server 510 that interfaces with multiple KAMDs and stores, analyzes and processes information from each KAMD. In another embodiment, the server 510 may be a dedicated machine that operates in conjunction with a single KAMD. In yet other embodiments, the KAMD and the server may be integrated into a single component such as a hand-held device or a desktop device with a measurement wand.

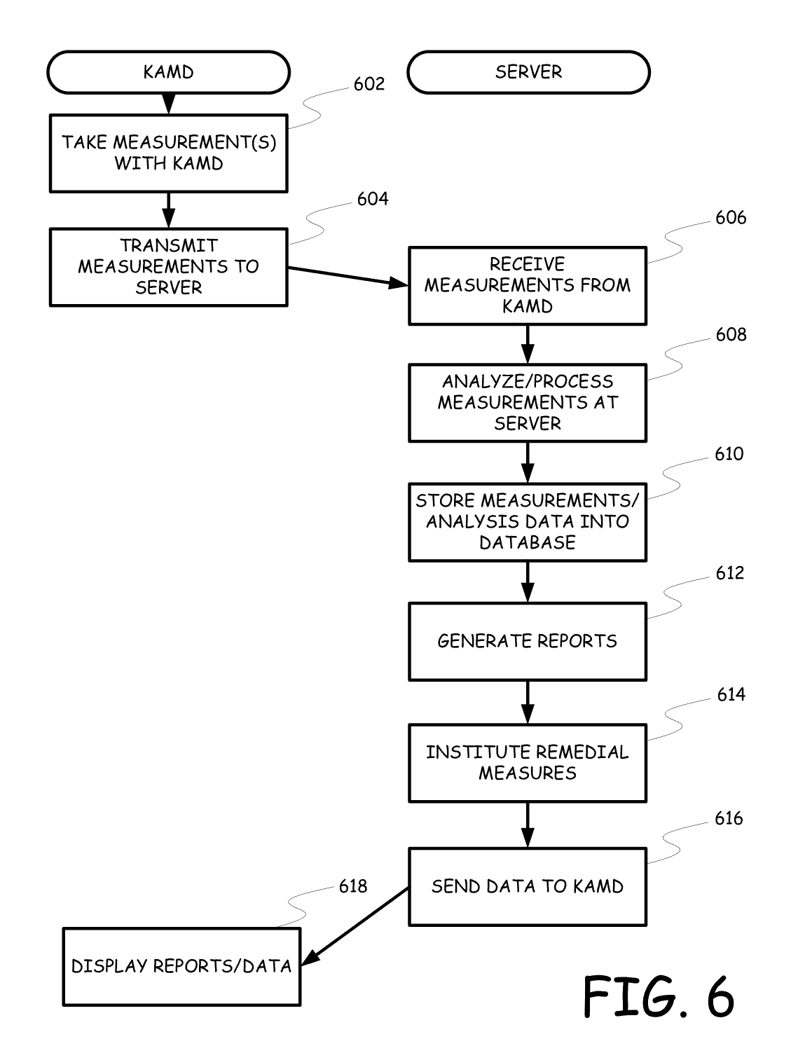

FIG. 6 is a flow diagram illustrating the exemplary flow of operation for an embodiment of the KMS. The various embodiments of the KMS operate to take kyphosis measurements of a patient, analyze the measurements and determine the level of kyphosis in the patient. Various measurement techniques may be employed utilizing the various embodiments of the KMS, but for purposes of illustration, the three point measuring approach in which angle measurements are taken at the sacral midpoint, at the juncture of the L1 and T12 vertebrae and a the juncture of the T1 and C7 vertebrae will be described. Utilizing the KAMD 520A, a measurement is taken at each of the three points of a patient's spine 602. These measurements are then either automatically, or in response to the user actuating the KAMD 520A to do so, transmitted 604 to the server 510. The measurements may be taken and transmitted one at a time, or each of the measurements can be taken and then transmitted. In addition, it will be appreciated that the KAMD 520A can operate in an online mode or an off line mode. In the online mode, the data measurements can be communicated to the server at anytime, whereas in the off line mode, the measurements are retained in the KAMD 520A until the device switches to the online mode of operation.

The transmission of the measurements may include an ID that is associated with a particular KAMD and/or a user or patient ID to identify the patient to which the measurements pertain. A variety of transmission techniques and protocols may be used for the transmission of this data, including encryption, data compression, data redundancy techniques as well.

Once measurements for a particular patient are received 606 by the server 510, the server 510 can analyze and/or process the measurements 608. The server 510 then stores 610 the measurement data, along with any analysis data into the database 515. The server 510 may then be further invoked or may automatically operate to generate reports 612, such as status and progress reports for particular patients, data collection reports for particular KAMDs (to check for calibration, aging, maintenance, accuracy etc.), data for multiple patients to generate comparison reports, etc. In addition, the server may be invoked or may automatically operate to institute remedial measures 614. For instance, the server may operate to make a recommendation for the patient to wear a back brace, or the server may provide recommended adjustments or settings for a patients back brace or other equipment.

The server may also send reporting and remedial measures information 616 back to the KAMD and the KAMD can display and/or provide access of the information 618 to the user of the device.

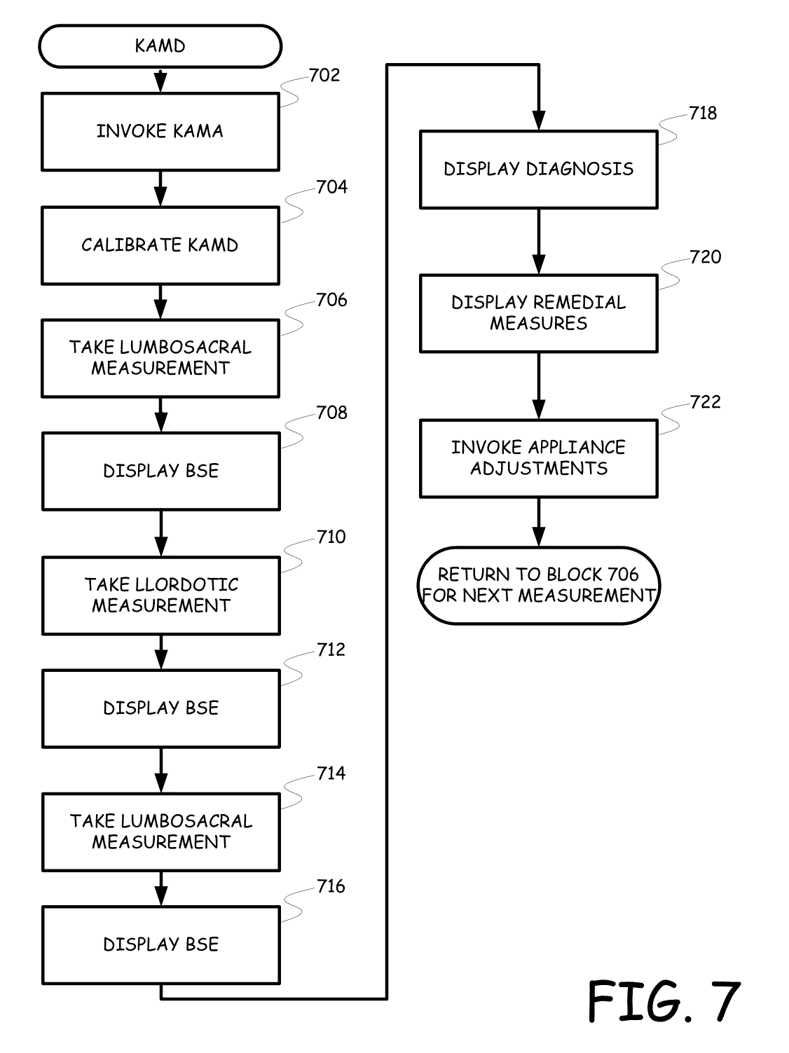

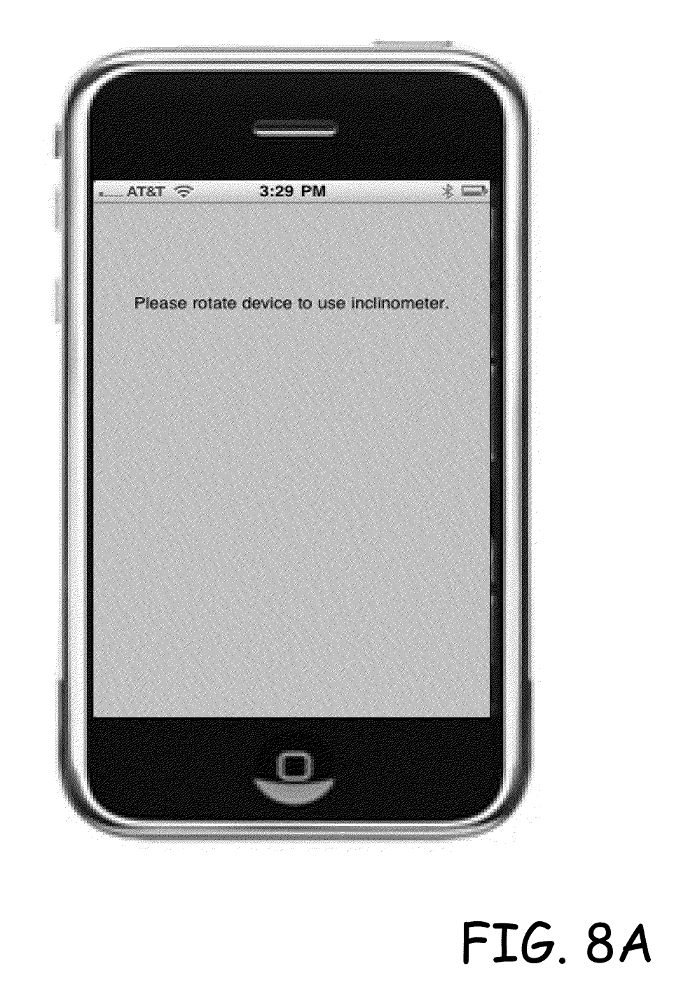

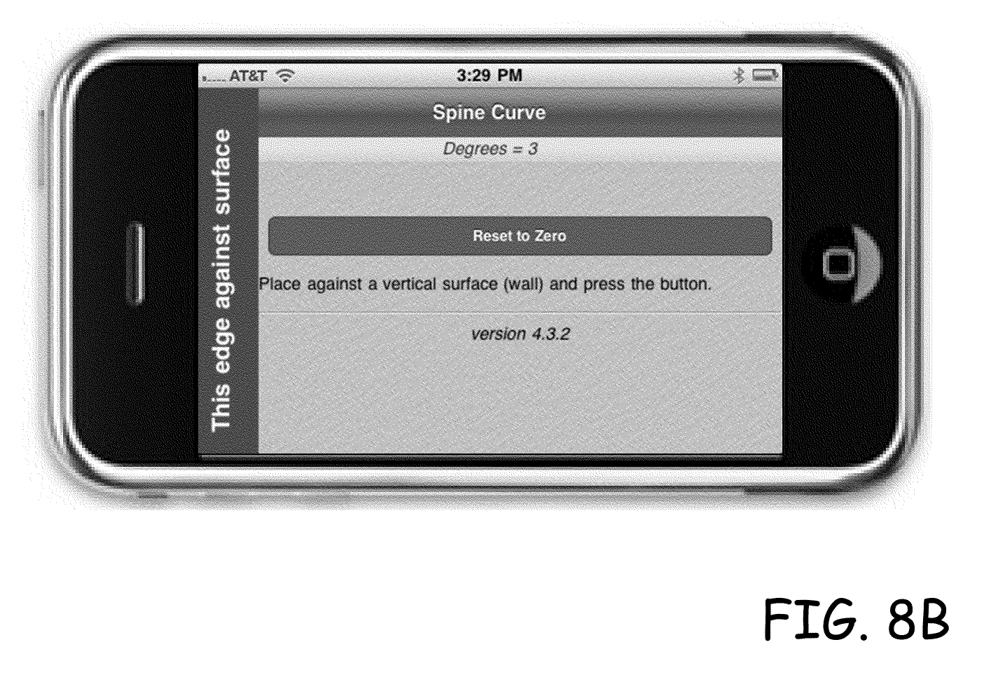

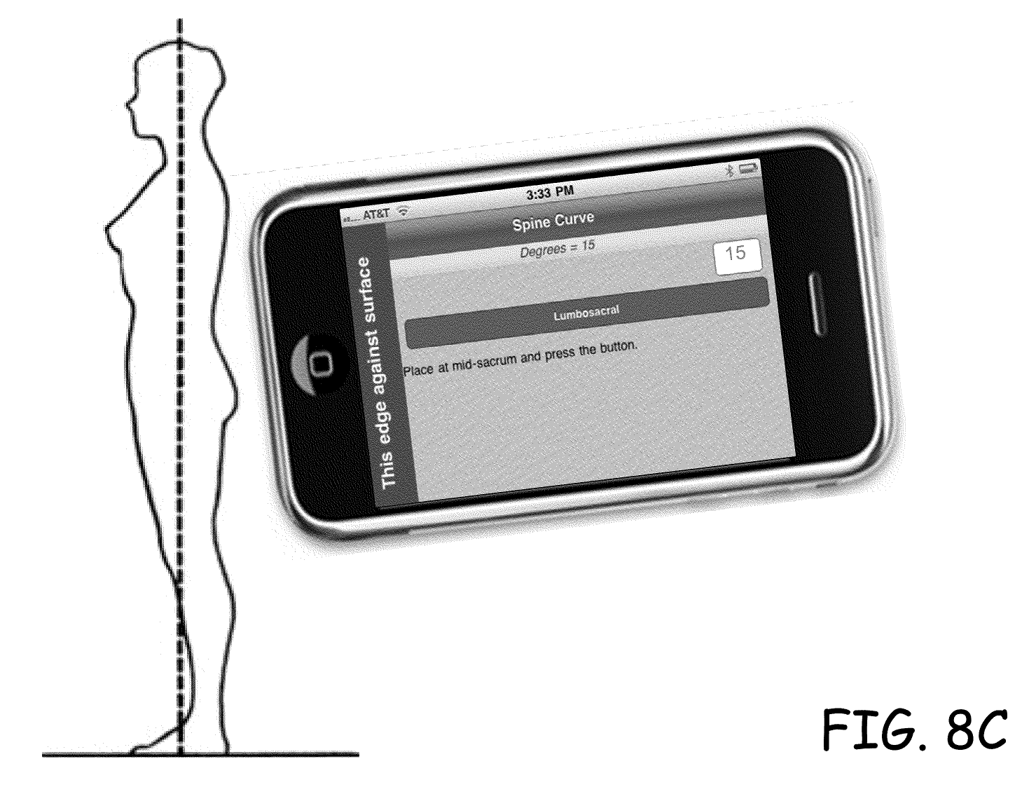

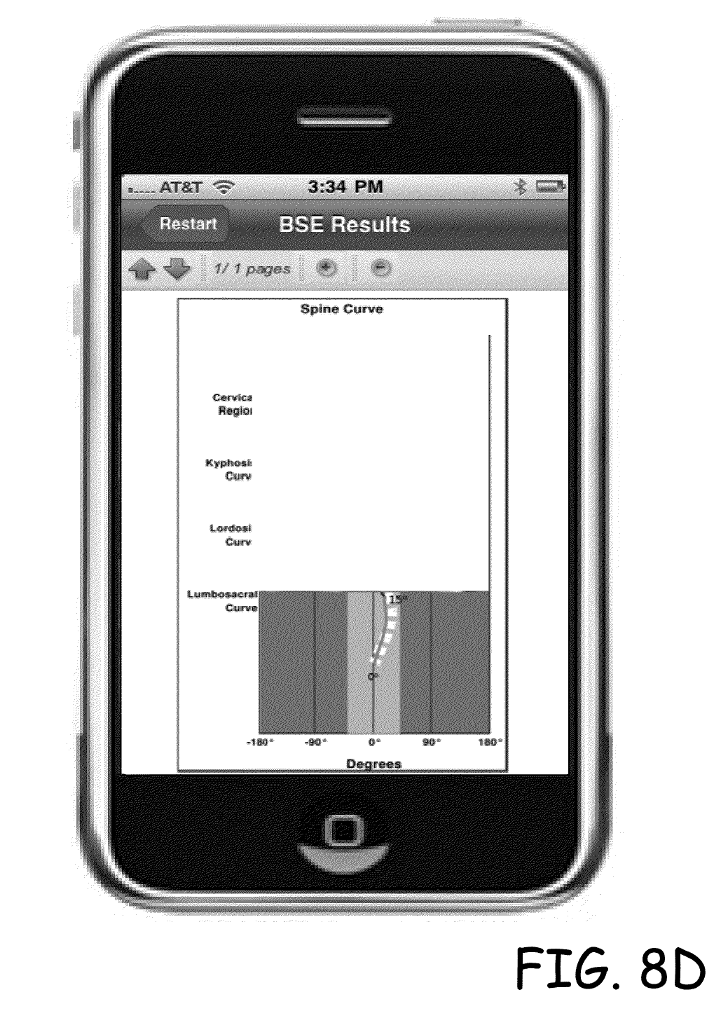

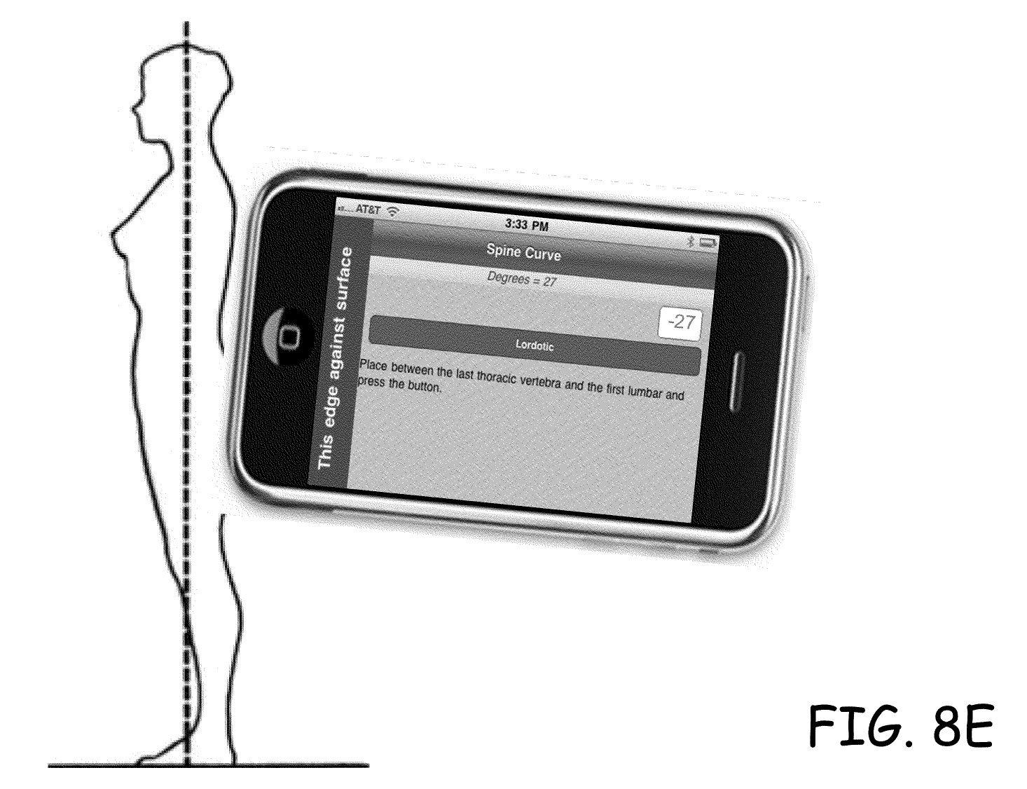

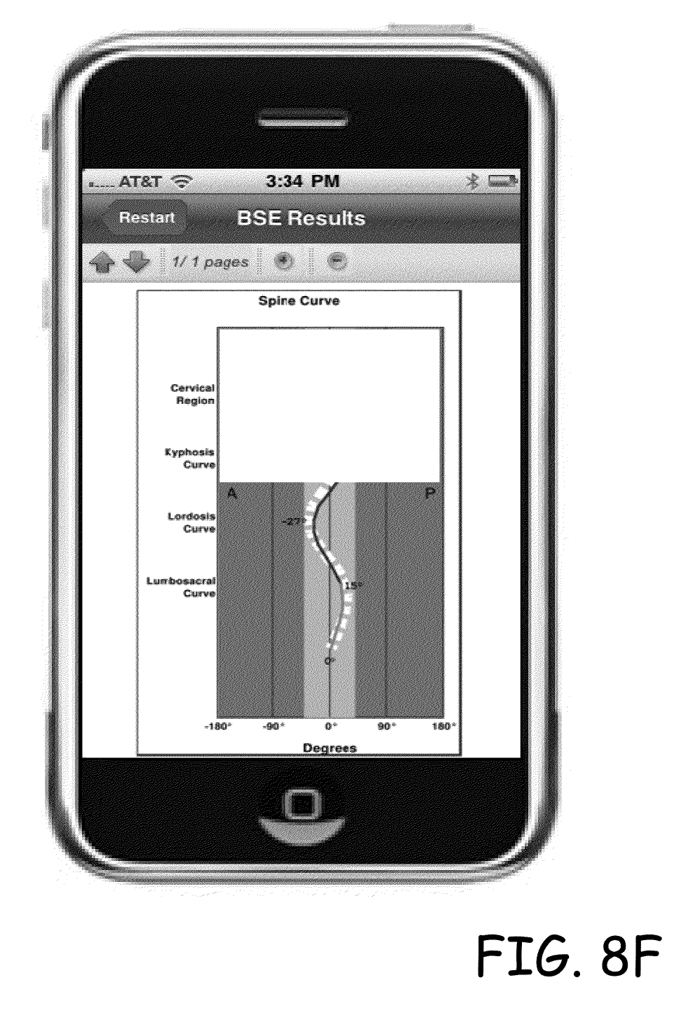

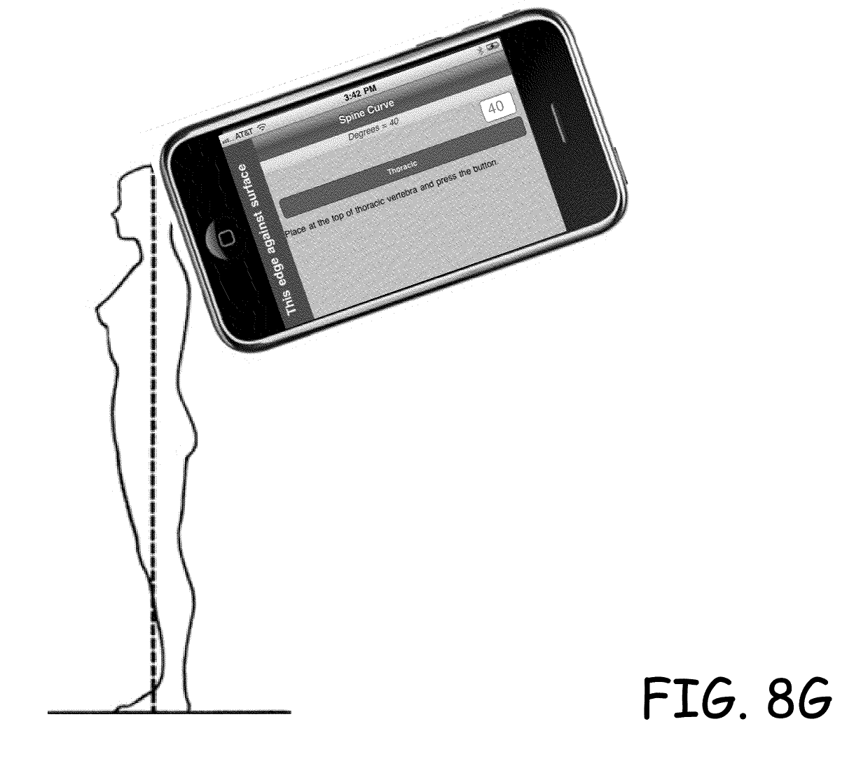

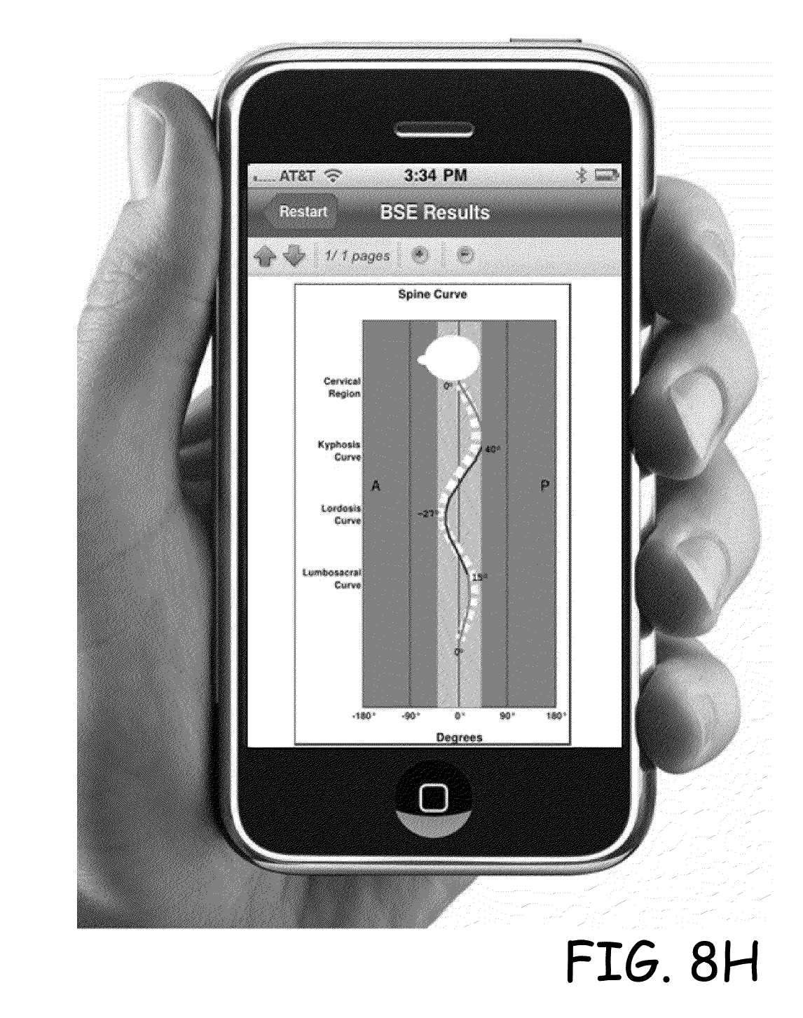

FIG. 7 is a flow diagram illustrating the operation of an exemplary embodiment of a KAMD. The exemplary embodiment of the KAMD will be described in terms of a kyphosis angle measurement application (KAMA) running on an IPHONE platform; however, it will be appreciated that any other mobile device or smart phone, commercially available, OEM or customized device could also be utilized. The flow diagram in FIG. 7 corresponds with the screen diagrams presented in FIGS. 8A-8H. Reference to the applicable screen shots in FIGS. 8A-8H is provided in conjunction with the description of the appropriate action block of FIG. 7. The KAMD, which includes the KAMA, operates as an inclinometer to make angle measurements of a patient's spine and then stores and/or transmits the measurements to a server. It will also be appreciated that the KAMA may also include functionality to analyze the measurements, present a report concerning the kyphosis status of the patient, generate a diagram showing the spine curvature and/or provide analysis, diagnoses and remedial actions for the user to implement or the patient to take.

Initially, the KAMA is invoked 702 on the KAMD and the user is presented with an informational screen FIG. 8A indicating that the user should rotate the device to use the inclinometer. Once the device is rotated, the KAMA displays a screen FIG. 8B 704 requesting the user to calibrate the KAMD prior to taking measurements. In the illustrated embodiment, the user is instructed to place an edge of the KAMD against a surface, such as a vertical wall, and then actuate the “RESET TO ZERO” button. It should be appreciated that the KAMA may utilize any of the buttons available on the platform device as well as using soft buttons on a touch sensitive screen of the platform device. Thus, throughout this description, reference to pressing or actuating a button can refer to any of these options. In addition, voice activation may be included in the KAMD and as such, voice commands may be used to control the KAMD. This operation will calibrate the KAMD and zero the inclinometer at a known vertical.

Once calibration is completed, the user is instructed to place the indicated edge of the KAMD against the patient at the mid-sacrum location and to press the button to take the lumbosacral measurement 706 (see screen in FIG. 8C). The screen of the KAMD displays the angle measurement at the location and when the button is actuated, the angle measurement is stored for the lumbosacral measurement.

After taking the lumbosacral measurement, the KAMD may display the bone safety evaluation (BSE) results 708 immediately on the screen as shown in FIG. 8D for the user to see and evaluate. The KAMD may optionally be configured to display the BSE after completion of a measurement or, only upon request by the user. In addition, the KAMD may optionally be configured to display the BSE for a fixed period of time after taking the measurement or, indefinitely until the user actuates a button or takes another action.

Next the KAMD presents a screen to instruct the user to place the indicated edge of the KAMD against the patient between the last thoracic vertebra and the first lumbar and then press a button to take the lordotic measurement 710 (see screen FIG. 8E). The screen of the KAMD displays the angle measurement at the location and when the button is actuated, the angle measurement is stored for the lordotic measurement.

After taking the lordotic measurement, the KAMD may display the bone safety evaluation (BSE) results 712 immediately on the screen as shown in FIG. 8F for the user to see and evaluate.

Next the KAMD presents a screen to instruct the user to place the indicated edge of the KAMD against the patient at the top of the thoracic vertebra and then press a button to take the thoracic measurement 714 (see screen FIG. 8G). The screen of the KAMD displays the angle measurement at the location and when the button is actuated, the angle measurement is stored for the thoracic measurement.

After taking the thoracic measurement, the KAMD may display the bone safety evaluation (BSE) results 716 immediately on the screen as shown in FIG. 8H for the user to see and evaluate.

For each measurement taken by the KAMD, the measurement results may also be sent to the server 510 for storing and evaluating. In some embodiments, the KAMD is able to process the measurements and generate the BSE diagrams internal to the KAMD while in other embodiments, the measurements may be sent to the server 510 and the BSE diagrams may be sent to the KAMD for display.

Depending on the configuration of the KAMD, the user preferences and/or user actuations, the KAMD may then display diagnosis information regarding the kyphosis measurements 718, display remedial measures that should be taken to assist the patient in his or her treatment 720 and/or invoke appliance adjustments to any appliances, such as a back brace, that the patient may be utilizing 722. Processing may then return to block 706 to take the next measurement or, the KAMA can be shut down until the next use.

In some embodiments, rather than utilizing the three point measurement technique, the KAMD may be configured to allow the user to take a sweeping measurement. In this embodiment, the user is instructed to place an indicated edge against either the upper or lower extremity of the spinal measurement locations and then slowly move the device over the spine to the opposing measuring point of the spine. In such an embodiment, the KAMD may take periodic measurements while the KAMD is traversing the spine and then generate the curve of the spine based on the multiple measurements. In yet another embodiment, the user may be instructed to place the indicated edge against either the upper or lower extremity of the spinal measurement locations and then press a button. The user may then slide the KAMD to the next measurement location and then press a button again to indicate the second measurement location. The user may then continue sliding the device to the third measurement point and again pressing the button when the device reaches the third measurement point. This technique combines the three point measurement technique along with the sliding technique in that the user provides positive notification when the measuring device reaches each of the three measuring points but, additional data samples can be taken as the device slides between the measuring points. Other embodiments may include sound feedback tones to assist the user in establishing the rate for sliding the KAMD. In some embodiments, the user may also make a measurement of the top of the spine and the bottom of the spine to establish the overall spinal length. Such measurements may be taken with a KAMD that is equipped to detect movement of the KAMD in the vertical plane and to determine the altitude or vertical height of the KAMD. In other embodiments, a measuring rule can be used and the KAMD may request the user to enter the overall spinal length into the device.

In some embodiments, as presented above, the KMS may operate to provide remedial measures in response to receiving and evaluating the spinal measurements. The remedial measures may be provided in the form of instructions that are displayed for the user and, the user can implement the instructions. Such instructions may include, as non-limiting examples, adjustments to be made to a back brace, a traction system, exercises to be performed, etc. In other embodiments, the remedial measures may actually cause signals to be sent to an appliance or apparatus, such as a back brace, a traction machine, or the like, and cause automatic adjustments to be made.

In one particular embodiment, the KMS includes an adjustable back brace that has one or more inclinometers embedded therein. In such an embodiment, the back brace operates as the KAMD and periodically can take angle measurements of the patient's spine. By placing an inclinometer at desired measuring points within the back brace, the inclinometers can periodically take angular measurements of the spine. Thus, the back brace KAMD may then automatically send these measurements to the server for analysis or, the back brace KAMD may include the hardware and/or software to analyze the measurements autonomously. In either case, the measurements can be used to identify diagnostic information, generate BSE reports and/or generate remedial measures. The remedial measures can be used to determine adjustments to be made to the back brace, which adjustments can be manually performed by the patient or a user, or the adjustments can be automatically invoked. For instance, the sever may send commands to the back brace KAMD to cause adjustments in the back brace KAMD. These adjustments may include, as non-limiting examples, the tightening or loosening of adjustments, inflating or deflating of air pockets in the back brace KAMD, etc.

Advantageously, the KAMD, as presented herein, allows periodic measurements of the patients spine to be taken and reported to the server, either immediately upon being taken or at a later time. Because the KAMD is portable in many embodiments, these measurements can be taken by the patient or a person assisting the patient while the patient is away from the physician's office. Thus, non-medical personnel can be trained to utilize the KAMD and as a result, the physician can obtain a large amount of progressive date related to the patient's kyphosis without requiring the patient to come to the physician's office. Further, for embodiments in which the back brace KAMD is utilized, the measurements can be taken automatically without requiring the patient or someone assisting the patient to take the measurements, thus completely eliminating any training requirements.

In addition, rather than automatically invoking remedial measures, the diagnoses and analysis of the angle measurements can be presented to a physician. The physician can then review the data to determine if any remedial measures are required. Thus, the physician may then request the patient to come to the office in order to have the remedial measures implemented. Further, in embodiments in which the patient is utilizing an apparatus that can be automatically adjusted, the physician may send adjustment information to the apparatus to automatically invoke the necessary remedial actions without requiring the patient to visit the physician's office. The automatic adjustments can be implemented in a variety of manners. For instance, the apparatus may be equipped to receive signals over the air. In other embodiments, the apparatus may interface to a wifi network and receive an email message containing adjustment information. Those skilled in the art will appreciate that other mechanisms may also be employed.

Thus, various embodiments of a KMS have been presented and described. It should be appreciated that the KMS may be distributed among various components as illustrated in FIG. 5, or the KMS may be entirely embedded within a mobile device, such as a smart phone equipped with functionality to detect movement and angular position of the device. For instance, the IPHONE developed and sold by APPLE, INC., includes accelerometers, compass gyroscope (such as a microelectromechanical (MEMS) gyroscope as provided in the IPHONE 4) and other technology to identify the location, position and movement of the device. For instance, the IPHONE technology enables the ability to detect six axes of motion. Other mobile devices, smart phones and other devices may similarly be used in embodiments of the KMS. For instance, the handheld remote devices utilized to control WII applications could also be utilized. Further, the KMS could be implemented within a NINTENDO WII, XBOX or other similar devices.

The various embodiments of the KMS can receive and store periodic measurements of the spinal angles of a patient and create a database of the measurements to show progression or digression over time. Advantageously, having the KAMD interface directly to the server eliminates human errors that may be invoked due to data entry mistakes. Further, the KMS advantageously allows remedial measures to be taken to accelerate progression or decelerate digression and then to easily observe the results of the remedial measures by monitoring future measurements and comparing them to previous measurements.

Another advantage of various embodiments of the KMS is that the user that is taking measurements of a patient, such as a physician, physicians assistant, nurse, care taker, etc., can access past data related to previous measurements. The KAMD may access this information internal to the KAMD and/or access the data on the server. Thus, the KAMD can present an analysis of the progression of kyphosis over time or, show the results of therapeutic activity. For instance, the user can compare measurements taken after wearing a back brace for a period of time with measurements taken prior to use of the back brace.

### Other Applications

It should be appreciated that the embodiments presented herein may be utilized in other applications as well. As an example, a KAMD may be specifically constructed to be suitable for measuring the placement of teeth within a patient. For instance, if the KAMD included a smaller profile, the user could place the edge of the KAMD on one tooth at a time to measure and report the position of a tooth in the patient's mouth. Such an application can be instrumental in the field of orthodontics. The patient equipped with the KAMD, could periodically take measurements of his or her teeth, or certain target teeth. These measurements could be transmitted to the orthodontist for evaluation. Based on the information, the orthodontist can track the progression of movement of the teeth between office visits and adjustments. In addition, as is known in the art, some of the orthodontic appliances include self adjustments that can be performed by the patient. Utilizing an orthodontic KMS, the orthodontist can more accurately provide instructions to the patient between visits as to adjustments that can be made.

Various applications may also be utilized in construction arts. For instance, curvature on load bearing walls or other structures can be taken with the KAMD and relayed to a server for analysis.

Those skilled in the art will appreciate that many other applications of this technology may also be employed and the present disclosure anticipates such uses.

In the description and claims of the present application, each of the verbs, “comprise”, “include” and “have”, and conjugates thereof, are used to indicate that the object or objects of the verb are not necessarily a complete listing of members, components, elements, or parts of the subject or subjects of the verb.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

It will be appreciated by persons skilled in the art that the present invention is not limited by what has been particularly shown and described herein above. Rather the scope of the invention is defined by the claims that follow.

What is claimed is:

1. An apparatus for measuring and analyzing spinal angles, the apparatus comprising:
a detector that identifies the position of the apparatus;
a calibrator that when actuated, calibrates the position of the apparatus to a known orientation;
a user interface executed by a processor in the apparatus that is configured to instruct a user to:
calibrate the apparatus;
take a first spinal angular measurement at a first location; and
calculate the curvature of the spine at the first location based on the first spinal angular measurement.

2. The apparatus of claim 1, wherein the user interface of the apparatus is further configured to instruct the user to:
take a second spinal angular measurement at a second location;
calculate the curvature of the spine at the second location based at least in part on the second spinal angular measurement.

3. The apparatus of claim 2, wherein the user interface of the apparatus is further configured to instruct the user to:
take a third spinal angular measurement at a third location;
calculate the curvature of the spine at the third location based at least in part on the third spinal angular measurement.

4. The apparatus of claim 3, wherein the user interface is further configured to display a bone safety evaluation based on the first, second and third spinal angular measurements.

5. The apparatus of claim 4, wherein the bone safety evaluation includes a graphical representation of the curvature of the spine.

6. The apparatus of claim 3, wherein the apparatus interfaces to a server and transmits the first, second and third spinal angular measurements to the server.

7. The apparatus of claim 6, wherein the apparatus is configured to receive a bone safety evaluation from the server and to display the received bone safety evaluation.

8. The apparatus of claim 3, wherein the apparatus is further configured to analyze the spinal angular measurements and generate remedial actions based at least in part on the spinal angular measurements.

9. The apparatus of claim 1, wherein the apparatus interfaces to a server and transmits the first spinal angular measurement to the server.

10. The apparatus of claim 9, wherein the apparatus is configured to receive a bone safety evaluation from the server and to display the received bone safety evaluation.

11. The apparatus of claim 1, wherein the detector includes a gyroscope.

12. The apparatus of claim 1, wherein the detector includes accelerometers.

13. The apparatus of claim 1, wherein the detector includes a gyroscope and one or more accelerometers.

14. A system for measuring and analyzing kyphosis in a patient, the system comprising:
a measuring device that can detect the angular position of the device when held against a surface of a spine;
a server that is communicatively coupled to the measuring device and is configured to receive measurements from the measuring device and store the measurements;
a user interface running on the measuring device and instructing a user to take measurements at specific locations of the spine; and
the system, in response to receiving spinal measurements, is configured to analyze the measurements and identify the curvature of the spine.

15. The system of claim 14, wherein the system generates a bone safety evaluation and displays a graphical image of the curvature of the spine on a display of the server.

16. The system of claim 14, wherein the system generates a bone safety evaluation and transmits information to the measuring device, the measuring device in response to receiving the bone safety evaluation information renders a graphical image of the curvature of the spine on a display.

17. The system of claim 14, wherein the system evaluates the measurements and presents remedial measures to be taken.

18. The system of claim 14, wherein the system synthesizes measurements taken of a patient over a period of time and presents a diagnosis regarding the progression of kyphosis in the patient.

19. The system of claim 18, wherein the system presents remedial measures to be taken to retard the progression of the kyphosis.

20. The system of claim 19, wherein the system interfaces with an apparatus used to control the progression of kyphosis and the system sends commands to automatically adjust the apparatus in view of the remedial measures.
